(19) United States
(12) Patent Application Publication
(10) Pub. No.: US 2011/0208104 A1
(43) Pub. Date: Aug. 25, 2011

(54) GARMENT SERVING TO COMBAT CELLULITE AND/OR VENOUS AND LYMPHATIC INSUFFICIENCY

(76) Inventor: Dominique Sellier, Paris (FR)

(21) Appl. No.: 13/100,676

(22) Filed: May 4, 2011

Related U.S. Application Data
(63) Continuation of application No. 11/502,898, filed on Aug. 10, 2006.
Application No. 11/502,898 is a continuation of application No. PCT/FR2005/000292, filed on Feb. 9, 2005.

(30) Foreign Application Priority Data
Feb. 11, 2004 (FR) 0401341

(51) Int. Cl. A61L 15/00; A41D 13/00

(52) U.S. Cl. 602/75; 2/69

(57) ABSTRACT

The garments and method disclosed herein serve to combat cellulite and/or venous and lymphatic insufficiency in at least one desired area of a lower and/or upper member of the body. The garments preferably includes an elastic knitted shell having an inner face and an outer face adapted to restrain with a degressive rate of pressure of the distal extremity of the member toward its proximal extremity, and a projection structure placed on a portion of the inner face corresponding to said area. The projection structures are capable of locally modifying the curvature of the knitted shell, or other material, when the garment is worn thereby increasing the compression exerted by the garment in said area.

### CROSS-REFERENCES TO RELATED APPLICATIONS

This application is a Continuation application of U.S. patent application Ser. No. 11/502,898 filed Aug. 10, 2006, which is a Continuation application of PCT Application Number PCT/FR2005/000292, filed on Feb. 9, 2005 for GARMENT SERVING TO COMBAT CELLULITE AND/OR VENOUS AND LYMPHATIC INSUFFICIENCY, which application is incorporated herein by this reference thereto, and which claims foreign priority to FR 0401341, filed Feb. 11, 2004. The disclosure of the prior applications are incorporated herein by reference.

### BACKGROUND OF THE INVENTION

### 1. Field of the Invention

This invention concerns wearable materials and items, and in particular garments, intended to help the wearer fight cellulite and possible venous and lymphatic sufficiency, and to methods for using the garments disclosed herein, as well as to methods for making the disclosed garments and components thereof.

### SUMMARY OF THE INVENTION

Several garments have been proposed to treat cellulite which are usually activated by sweating. It is, today, acknowledged that sweating does not overcome hypertrophy of fatty tissues.

Cellulite affects 95% of women, both slim and corpulent. Cellulite or lipoedema corresponds to a hypertrophy of the fat lodged just under the skin in the hypodermis, associated with retention of fluids and toxins. Whereas, excess weight is due to the storage of fats at a deeper level.

Causes of cellulite are numerous. They could be endogenous in origin with a genetic predisposition, poor blood circulation and also hormonal variations (menstruation, pregnancy, menopause). They could also be due to exogenous factors such as a sedentary life style in particular, lack of physical activity and also stress and anxiety.

Although there are several causes and the patho-physiological symptoms are still poorly known, there are two main factors that contribute to the worsening of cellulite [0008] firstly the increase in size of the fat cells (adipocytes) due to storage of fats linked essentially to an unbalanced diet and sedentary life style [0009] and secondly the increase in water infiltration in the tissues, related to poor venous and lymphatic blood flow back to the heart.

A study involving the biopsy of 48 womens' thighs affected by cellulite revealed a constant dilatation of the fatty tissue venules visible both in the capillaries of the dermis and hypodermis, associated with storage of fats in the fatty tissues. The same vein alterations were also noticed by infrared thermography that revealed a colder skin temperature in the cellulite areas.

As the fatty tissues increase in size, the process of self nourishment with a deterioration of blood circulation and stagnation of toxins is noticed. The connective supporting tissue loses its elasticity and becomes fibrous. At an advanced stage, the cellulite becomes fibrous which gives the skin a dimpled and “orange peel” appearance. There are three clinical stages of “orange peel” skin. [0012] stage 1: the dimpled appearance is visible when the skin is pinched [0013] stage 2: the dimpled appearance is visible when standing [0014] stage 3: the dimpled appearance is permanently visible in all positions

The effectiveness of treatment proposed can be assessed through high frequency ultrasound exploration by measuring the thickness of the subcutaneous tissue and infiltration of water in the tissues.

Lack of physical exercise and a sedentary life style promote the storage of fats. Muscles represent 20 to 25% of womens' bodies and since muscles consume energy even at rest, it is important to take care of ones muscles and limit their involution with age. At the age of 65, a person loses 30% of the muscles that he/she has at 25. Regular physical exercise has a slimming effect in the long term, reducing insulin production by the pancreas and also helps to stave off hunger. Furthermore, the fats seem to dissolve notably in areas where the muscles are stimulated through exercise.

Medical experts are in consensus about the benefits involved in graduated compression associated with walking in venous-lymphatic insufficiency. Furthermore the beneficial effects of contraction of the calf muscles by activating what is called the “muscular pump of the calf” may result in better blood flow from the lower limbs back to the heart.

The Inventor has reported that due to the graduated compression (on average the pressure decreases, in a relation of about 40-50% between the ankle and the calf) and Laplace's Law, a compression stocking does not really act effectively at the level of the thigh and the pelvis.

According to Laplace's Law, the external pressure (P) exerted by an elastic casing, shell, sheath or similar material, particularly a stocking on a desired area of the body, is directly proportional to the tension (T) of the elastic casing, sheath, shell or similar and inversely proportional to the curvature radius (r) of the casing, sheath, shell or similar in the mentioned area. This law is expressed by the formula P=T/r

Where (P) represents the pressure (g/cm2) exerted on the skin, (T) the tension (g/cm) of the elastic fabric and (r) the curvature radius (cm) of the compressed area.

Laplace's Law is only valid for elastic fabrics, the term fabric used as generally accepted to include knitted and woven fabrics and similar materials. For a fabric having a constant tension (given), the pressure decreases when the curvature radius of the limb increases.

Consequently the Inventor has reported that at the upper one third part of the thigh, the pressure exerted by compression stockings is no longer effective on the thigh muscles, on account of the diminishing pressure and also because of the consequences of Laplace's Law, since this part is almost flat; the standard compression stockings do not exert any efficient pressure here. Since the curvature radius of the lower limbs varies according to each part considered, it is very difficult to measure pressure in vivo. In fact, research and testing laboratories define the pressure exerted by compression stockings, by calculating the pressure exerted on a cylinder with a well-defined radius based on the characteristics of an elastic knitted fabric.

It is possible to increase the elastic compression at the thigh, but this would make it difficult to put on the garment and furthermore, difficult to wear, as it would be anti-physiological in relation to the venous and lymphatic blood flow back to the heart.

The Inventor has also reported that manual lymphatic drainage disposes of excess fluids in the tissues and evacuates waste from cellular metabolism. This drainage occurs through an initial process called “collection” carried out by the network of lymphatic capillaries located in the infiltrated areas. The “collection” results from the localized increase in the tissue pressure through massage; the greater the pressure, the higher the removal of excess fluids by the lymphatic capillaries.

The second process of drainage consists in the “evacuation” of the fluids and toxins by the lymphatic capillaries, far from the infiltrated area, towards the pre-collectors followed by the lymphatic collectors, of which the major trunk is represented by the satellite collectors of the saphenous vein.

The instant invention is directed to wearable materials and items such as garments and methods which act on the different mechanisms of self nourishment of cellulite resulting in orange peel skin, and which are particularly effective concerning localized micro circulation, the collection of toxins and fluids and venous and lymphatic macro circulation.

This goal is perfectly achieved by the garments and methods disclosed herein, which are intended to fight cellulite and/or venous and lymphatic insufficiency in at least one desired area of a lower and/or upper limb. These wearable materials, items or garments preferably comprise a knitted elastic sleeve, casing, shell, sheath or similar s material presenting an inner and an outer side. The sleeve, casing, shell, sheath, or similar, may be continuous or may be non-continuous on the desired area, e.g. the sleeve, casing shell, sheath or similar may have open areas where a portion of the wearer's skin on an area is uncovered, or areas comprising a different fabric or material.

Preferably, the knitted casing, sleeve, shell, sheath or similar has a compression effect with a diminishing degree of pressure from the distal end of the aforesaid limb to its proximal end and comprises projection structures, protuberances or a bumpy composition (also referred to herein as ‘bumpy aspect’), on the inner side of the garment corresponding to the desired area. The aim of the bumpy aspect, projection structures, protuberances or bumpy composition is to change the curvature of the knitted garment locally when worn; it is designed in a way which increases the compression exerted by the garment in the aforesaid desired area, in application of Laplace's Law. It is through this combination of diminishing compression, but localized increase in compression brought about by the bumpy aspect, projection structures, protuberances or bumpy composition of the garment that the goal is achieved.

The garment according to the invention stimulates localized micro circulation along with collection of toxins and fluids by means of the bumpy aspect, projection structures, protuberances or bumpy composition fitted in the parts of the garment corresponding to the cellulite area. Moreover venous and lymphatic macro circulation is also stimulated because of graduated compression. The bumpy aspect, projection structures, protuberances or bumpy composition on the inner side of the garment causes an increase in pressure on the subcutaneous tissue by changing the curvature radius locally without increasing the elastic compression at the proximal end of the upper and lower limbs. Such an increase would not be effective as it would undermine the diminishing action of elastic compression.

The bumpy composition, or the bumpy aspect, of the garment is preferably made up of several protuberances or projection structures (which will be used interchangeably herein). This effect can be obtained through printing on the knitted fabric with raised deposits. It can be obtained at the time of knitting with the formation of raised patterns in accordance with various well-known techniques such as “knitted double fabric” for example so that the bumpy composition or bumpy aspect can be preserved even after the garment is worn and the fabric stretched.

According to the areas to be stimulated and following the results sought, the bumpy composition, or bumpy aspect, may be in varying numbers and sizes. Since there are preferably several protuberances or projection structures, they are preferably placed at intervals to ensure proper airing of the area on which they are applied, so as not to increase heat and sweating. The protuberances or projection structures are preferably under six millimeters in height (H), and more preferably have a height (H) of about 0.5 to about 3 mm, and spacing of preferably about 1 to about 3 times the said height (H). The bumpy composition or bumpy, aspect can also contain active substances using micro encapsulation techniques, for cosmetic or hygienic purposes.

As previously stated, the bumpy composition or bumpy aspect of the inner side of the garment will contribute to a better collection of fluids and toxins in the infiltrated areas by causing the tissue pressure to increase. The bumpy aspect or bumpy composition is designed in the areas mainly concerned by cellulite preferably at the principle saphenous veins. Its action is enhanced during physical exercise particularly when doing sport, through a massaging effect which acts in combination with the pressure exerted. This combined effect of pressure exerted and massage in the cellulite affected area or areas will break down the cellulite by promoting fluid and toxin collection.

Furthermore, the bumpy composition, or bumpy aspect, on the inner side along the lymphatic collectors contributes to a better “evacuation” far from the infiltrated parts, of the toxins and fluids picked up by the capillaries towards the most important collectors, which will also be stimulated by variations in pressure during physical exercise.

A bumpy composition, or bumpy aspect, can also be designed on the outer side of the garment along the body areas mainly affected by cellulite in order to improve the pressure on tissues during physical exercise on the floor.

The garments may include in their application leggings or leotards for the lower limbs and shirts, such as a tee-shirt, for the upper part of the body. For the latter, a seam can be used at the armpits to allow for a gradient of pressure between the elbow and the shoulder.

Physical exercise plays an important role in tackling lipogenesis (increase of fat cells) due to sedentary life style. It is possible to improve the beneficial effects of physical activity, particularly sport, whether it be walking, cycling or a work out when this invention is worn. And even at rest, the wearer of this invention benefits from the advantages, and comfort of decreasing elastic compression.

### OBJECTS OF THE INVENTION

It is an object of the present invention to provide garments intended to help the wearer fight cellulite and possible venous and lymphatic insufficiency, and to methods for using the garments disclosed herein, as well as to methods for making the disclosed garments. These and other objects and advantages of the present invention will be apparent from a review of the specification and accompanying drawings.

### BRIEF DESCRIPTION OF THE DRAWINGS

FIG. 1 is a schematic representation of ankle-length leggings in accordance with a preferred embodiment of the invention, with a general front view showing the areas having a bumpy composition on the inner side.

FIG. 2 is a schematic representation of a preferred embodiment of the leggings in FIG. 1 seen from the back.

FIG. 3 is a partial schematic representation of a preferred embodiment of the leggings showing a bumpy composition on the outer side of the garment, seen from the front.

FIG. 4 is a partial schematic representation of a preferred embodiment of the leggings in FIG. 3 seen from the back.

FIGS. 5 and 6 show a cross view of a preferred embodiment of the protuberances on the inner side in an area which is respectively without protuberances on the outer side (FIG. 5) and with protuberances on the outer side of the elastic knitted fabric (FIG. 6).

FIG. 7 is a schematic representation of a preferred embodiment for a leotard for dancers according to the invention.

FIGS. 8 and 9 schematically represent a preferred embodiment for a tee-shirt according to the invention seen from the front (FIG. 8) and from the back (FIG. 9).

FIG. 10 shows a cross-sectional view of a preferred embodiment having the protuberances on the inner side of the material, when worn by a user.

### DESCRIPTION OF THE PREFERRED EMBODIMENT(S)

The detailed description set forth below in connection with the appended drawings is intended as a description of presently-preferred embodiments of the invention and is not intended to represent the only forms in which the present invention may be constructed and/or utilized. The description sets forth the functions and the sequence of steps for constructing and operating the invention in connection with the illustrated embodiments. However, it is to be understood that the same or equivalent functions and sequences may be accomplished by different embodiments that are also intended to be encompassed within the spirit and scope of the invention.

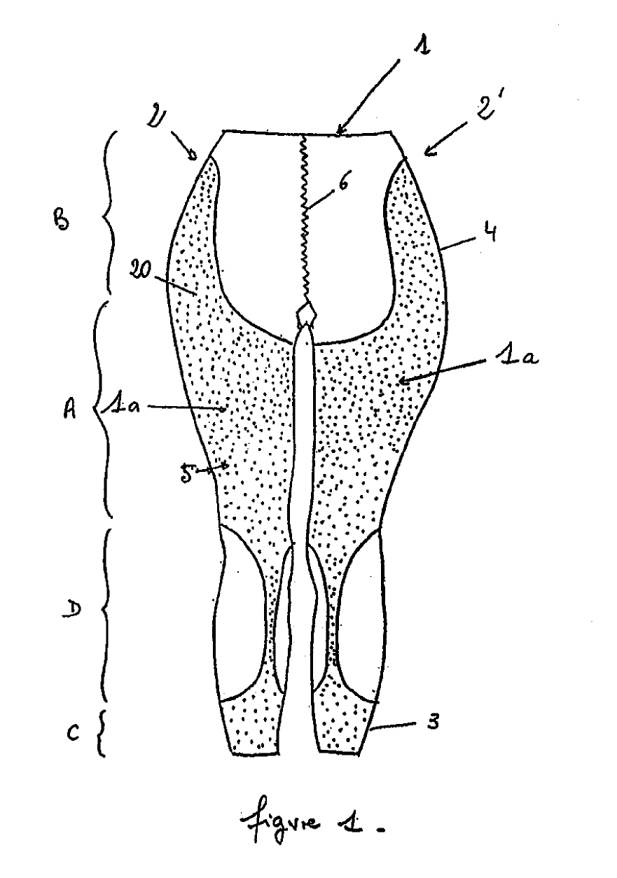

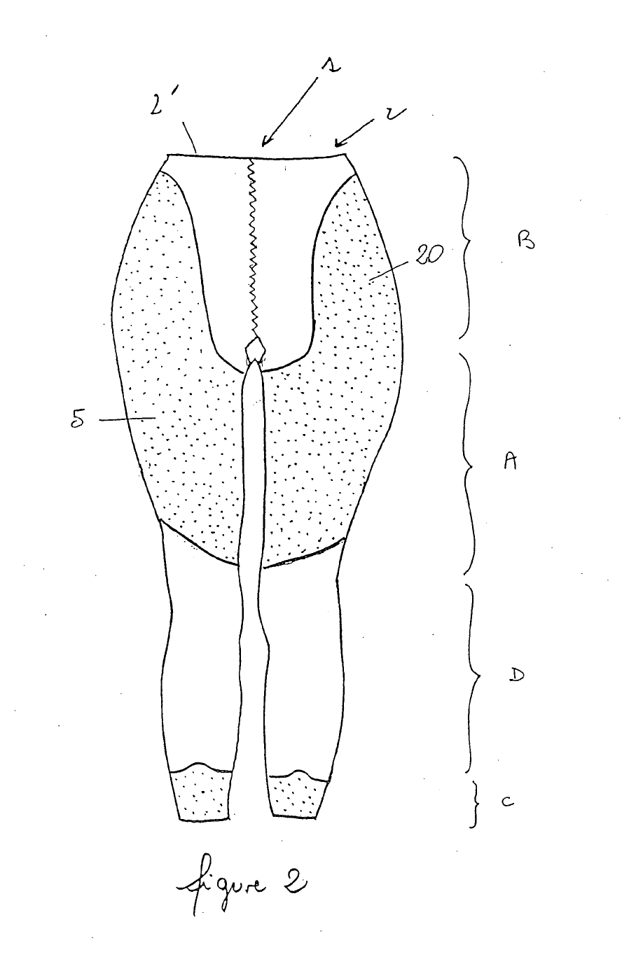

Turning to the appended figures, in FIG. 1, the garment accords one preferred embodiment of the invention as a pair of sports leggings (seen from the front in FIG. 1). Preferably, the leggings 1 are made up of two half-tights 2 and 2′ linked together by seam 6, each preferably being made on a straight-bar or circular knitting machine in accordance with well known techniques to obtain a diminishing gradient of pressure between areas or zones that are cellulite-prone or otherwise are to be treated, such as for example, a first zone 3 corresponding to the ankle and a second zone 4 corresponding to the upper thigh. The leggings 1 preferably do not cover the feet as can be seen in FIG. 1.

The compression values of leggings 1 may vary according to the effect desired. For example the compression values on anatomical zones of a person wearing leggings may be: TABLE-US-00001 Ankle pressure Leg pressure Thigh pressure (3) (7) (8) In mmHg 8-18 8-15 6-12

Any weaving or knitting process and all fabrics that provide the desired pressure values may be used. In the case of associated severe venous or lymphatic insufficiency, the usually desired differential compression of 50% is possible by fitting the garment with a compression device such as support knee-length stockings.

According to one aspect of the invention, this diminishing elastic compression from the distal to the proximal end of the limb is associated with a bumpy composition on the inner side and possibly the outer side of the garment, in order to combine improved venous and lymphatic circulation with an action of massage and pressure on the cellulite areas with a “collection” and “evacuation” effect.

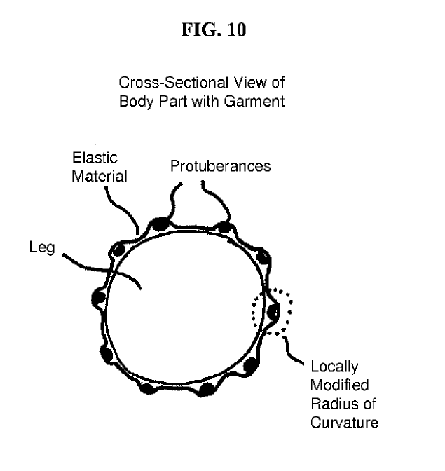

FIG. 10 illustrates an example of an aspect of the invention, in which the projection structures (protuberances) are provided on the inner side of the garment when the garment is worn on a leg for example. The radius of curvature of the elastic material is modified in the locations of the projections. By modifying the curvature radius, the pressure exerted on the skin changes in accordance with Laplace's law. Thus, the projection structures change the compression of the garment at their discrete locations.

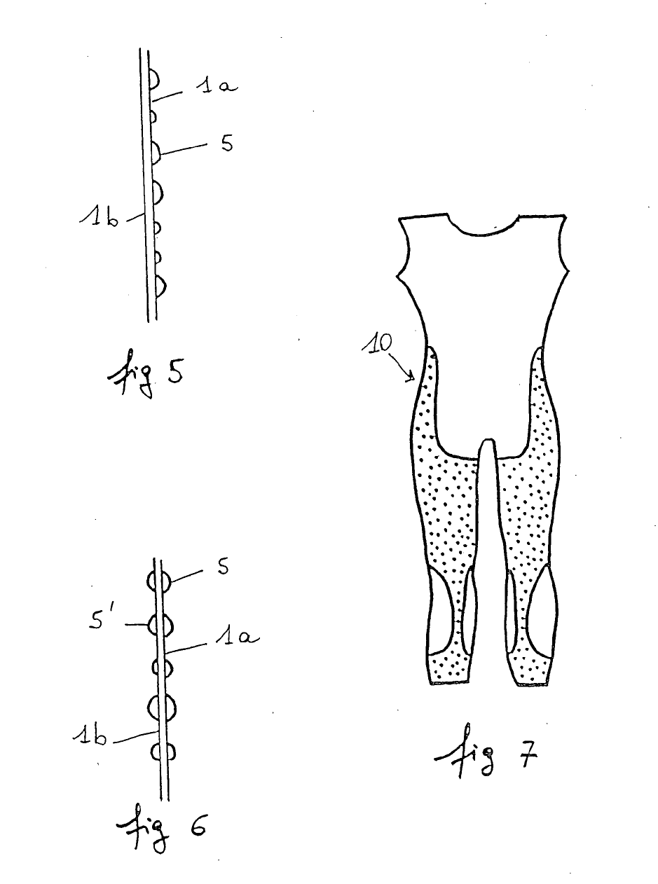

As a preferred alternative embodiment for creating the invention, FIGS. 5 and 6, the bumpy composition is presented as multiple protuberances, or projection structures, 5 and 5′ fixed respectively on the inner side 1a and outer side 1b of garment 1.

These protuberances 5 and 5′, preferably curved, hemispheric or half moon shaped, are preferably made from plastic material, preferably semi-rigid, such for example, in thermoplastic elastomer, silicone or composite. These protuberances can also contain active substances according to the technique of micro encapsulation, for cosmetic or hygienic purposes.

Preferably, the protuberances 5, 5′ have a diameter (D) of under about 8 millimeters and a height (H) preferably of about less than 6 millimeters. Their number and sizes, however, may be determined according to the areas requiring stimulation and effect desired.

The protuberances 5 on the inner side of the leggings are preferably placed according to the following third zone 20: the full circumference of the thighs (A), up to is above the knees, both sides of the hips (B), the circumference of the ankles (C) and on the inner side of the leg (D). Their beneficial action combined with diminishing compression occurs in these parts (A,B,C,D) marked with a dotted line on the figures, particularly during specific physical exercises concerning these areas or during speed walking or pedaling.

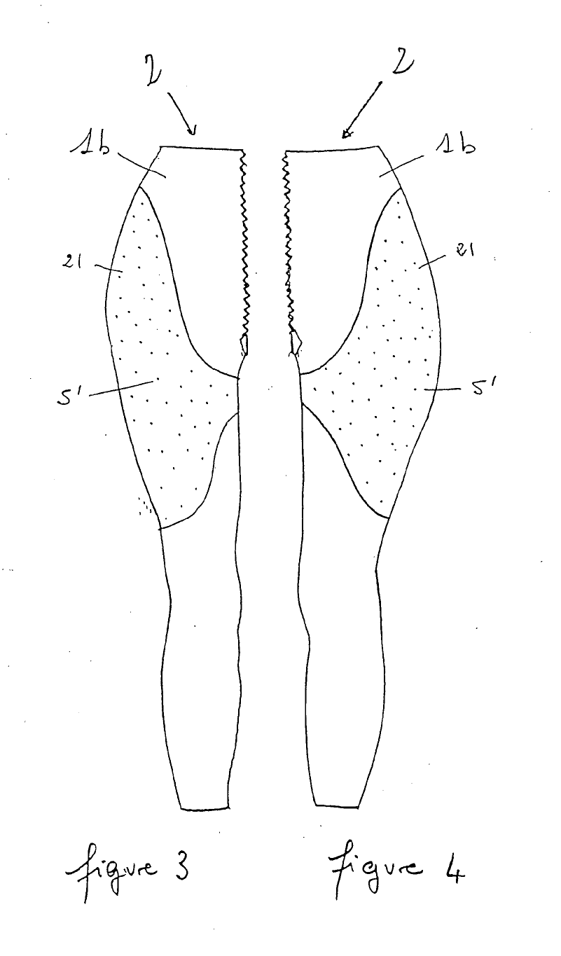

The protuberances 5′ on the outer side 1b of the leggings are represented in FIGS. 3 and 4. During floor exercises, the protuberances bring about an increase in the massage pressure exerted through a simple mechanical pressing action of the protuberances into the skin. In this case, the protuberances on the inner and outer side of the sport garment are preferably superposed to enhance this mechanical pressing action. The 5′ protuberances on the outer side 1b of leggings 1, are preferably fitted (FIGS. 3 and 4) solely in a fourth zone 21 corresponding to the circumference of the upper thigh and on the sides along the thighs and hips.

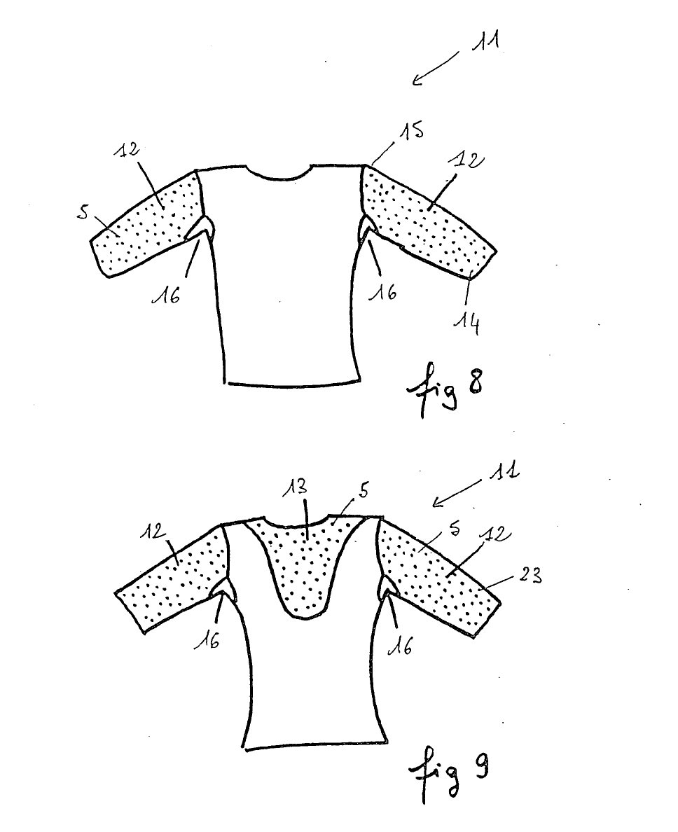

In another variation, (FIG. 7), garment 10 according to the invention, may be a leotard for dancers, in which the leggings preferably have a sleeveless extension covering the upper part of the body.

In another variation (FIGS. 8 and 9), garment 11, according to the invention, uses the principle of elastic compression associated with a bumpy composition on the inner side and possibly the outer side of a fifth zone 23 of arms 12. As illustrated in FIG. 9, protuberances 5 have preferably also been fitted on a part of the back 13 for massage action. A seam 16 is used at the armpits to obtain a pressure gradient between the elbow 14 and shoulder 15. These protuberances can also contain active substances, using micro-encapsulation technology, for cosmetic or hygienic purposes.

This invention is not limited to the methods of production described and illustrated above which are given as examples and are not exhaustive. The bumpy composition in the area or areas to be treated can be created in any appropriate form provided that the technical effect of localized increase in compression exerted by the garment is obtained through change in the radius of the knitted garment. This composition could be achieved at the time of knitting with the creation of a thicker knit, that is sufficiently rigid so that it continues to have a bumpy effect when worn during sport.

While the present invention has been described with regards to particular embodiments, it is recognized that additional variations of the present invention may be devised without departing from the inventive concept.

What is claimed is:

1. A wearable item to combat cellulite and/or the venous and lymphatic insufficiency in at least one desired area of the body, comprising:
an elastic material comprising an inner face and an outer face, said material adapted to restrain the area of the body with a diminishing rate of pressure from the distal extremity of the area of the body toward its proximal extremity; and
at least one projection structure placed on a portion of the inner face, said projection structure adapted to modify the curvature of the material to be worn whereby compression exerted by the material in the desired area of the body is increased to lessen cellulite and/or venous and lymphatic insufficiency.

2. The material as claimed in claim 1, comprising a wearable item incorporating plurality of projection structures having a substantially curved form.

3. The material as claimed in claim 1, wherein the projection structures comprise a material selected from the group consisting of plastic, semi-rigid plastic, thermoplastic elastomer, silicone and composites thereof.

4. The material as claimed in claim 1, wherein the projection structures are formed by impression, with deposits in relief.

5. The material as claimed in claim 1, wherein the material comprises a fabric and the projection structures are formed integral with the fabric at the time of knitting.

6. The material as claimed in claim 1, wherein the projection structures of the inner face are placed on the level of the parts of the material intended to be in contact with at least one zone to be treated and with the veno-lymphatic saphenous collector.

7. The material as claimed in claim 2, wherein the projection structures of the inner face are placed on the level of the parts of the material intended to be in contact with at least one zone to be treated and with the veno-lymphatic saphenous collector.

8. The material as claimed in claim 1, further comprising protuberances on the outer face and wherein a portion of protuberances on the outer face of the material are in superposition with a portion of the projection structures on the inner face of the material.

9. The material as claimed in claim 2, further comprising protuberances on the outer face and wherein a portion of protuberances on the outer face of the material are in superposition with a portion of the projection structures on the inner face of the material.

10. The material as claimed in claim 1, wherein at least one projection structure on the inner face contains micro encapsulated active substances.

11. The material as claimed in claim 1, wherein the material comprises a tee-shirt having armpits and wherein at least one of the armpits comprises a part of ease under the armpit in order to maintain the decreasing scale of pressure between at least one elbow and at least one shoulder of a wearer.

12. The material as claimed in claim 2, wherein the projection structures on the inner face of the material have a height (H) of less than about 6 mm.

13. The material as claimed in claim 12, wherein the projection structures on the inner face of the material have a height (H) of between about 0.5 mm and about 3 mm.

14. The material as claimed in claim 2, wherein the projection structures on the inner face of the material have a height and wherein the projection structures are spaced from one another at a distance of between about 1 to about 3 times the height (H) of the projection structures on the inner face.

15. A wearable material to combat cellulite and/or the venous and lymphatic is insufficiency in at least one desired area of the body, comprising:
a. an elastic sheath comprising an inner face and an outer face, said sheath adapted to restrain the area of the body with a diminishing rate of pressure from the distal extremity of the area of the body toward its proximal extremity;
b. at least one projection structure placed on a portion of the inner face, said projection structures having a height (H) of less than about 6 mm and adapted to modify the curvature of the sheath when the wearable material is worn; and
c. at least one protuberance on the outer face, whereby compression exerted by the wearable material in the desired area of the body is increased and wherein a portion of protuberances on the outer face of the wearable material are in superposition with a portion of the projection structures on the inner face of the wearable material.

16. The wearable material as claimed in claim 15, wherein the projection structures on the inner face of the sheath have a height (H) of between about 0.5 mm and about 3 mm.

17. The wearable material as claimed in claim 15, wherein the projection structures on the inner face of the sheath are spaced from one another at a distance of between about 1 to about 3 times the height (H) of the projection structures on the inner face.

18. A wearable material to combat cellulite and/or the venous and lymphatic insufficiency in at least one desired area of the body, comprising:
a. an elastic sleeve comprising an inner face and an outer face, said sleeve adapted to restrain the area of the body with a diminishing rate of pressure from the distal extremity of the area of the body toward its proximal extremity;
b. at least one projection structure placed on a portion of the inner face, said projection structures having a height (H) of less than about 6 mm and adapted to modify the curvature of the sleeve when the wearable material is worn; and
c. at least one protuberance on the outer face, whereby compression exerted by the wearable material in the desired area of the body is increased and wherein a portion of projection structures on the inner face comprises micro encapsulated active substances.

19. The wearable material as claimed in claim 18, wherein the projection structures on the inner face of the sleeve have a height (H) of between about 0.5 mm and about 3 mm.

20. The wearable material as claimed in claim 18, wherein the projection structures on the inner face of the sleeve are spaced from one another at a distance of between about 1 to about 3 times the height (H) of the projection structures on the inner face.
